(19) United States
(12) Patent Application Publication
(10) Pub. No.: US 2021/0275450 A1
(43) Pub. Date: Sep. 9, 2021

(54) SIMPLE POVIDONE-IODINE-LIDOCAINE GEL PREP FOR TRANSRECTAL ULTRASOUND PROSTATE NEEDLE BIOPSY AND METHOD OF PERFORMING AN ULTRASOUND PROSTATE NEEDLE BIOPSY USING SAME

(71) Applicant: Gary S. Fialk, Herndon, VA (US)

(72) Inventor: Gary S. Fialk, Herndon, VA (US)

(21) Appl. No.: 17/249,491

(22) Filed: Mar. 3, 2021

Related U.S. Application Data
(60) Provisional application No. 62/987,073, filed on Mar. 9, 2020.

(51) Int. Cl. A61K 9/06; A61B 17/34; A61K 9/00; A61K 31/4015; A61K 31/167; A61K 47/02; A61K 47/10; A61K 47/24; A61K 47/38

(52) U.S. Cl. CPC A61K 9/06; A61B 17/3403; A61K 9/0031; A61K 31/4015; A61B 2017/3413; A61K 47/02; A61K 47/10; A61K 47/24; A61K 47/38; A61K 31/167

(57) ABSTRACT

A combination gel lubricant composition for use in transrectal ultra-sound guided needle biopsy procedures comprising a 10% povidone iodine antiseptic gel and 2% lidocaine anesthetic gel to form the combination gel lubricant. The combination gel lubricant is used in a method of performing a transrectal ultra-sound guided needle biopsy.

### CROSS REFERENCE TO RELATED APPLICATION

This application claims the benefit of U.S. Provisional Patent Application Ser. No. 62/987,073, entitled “SIMPLE POVIDONE-IODINE-LIDOCAINE GEL PREP FOR TRANSRECTAL ULTRASOUND PROSTATE NEEDLE BIOPSY AND METHOD OF PERFORMING A PROSTATE NEEDLE BIOPSY USING SAME,” filed Mar. 9, 2020, the content of which is incorporated herein by reference.

### BACKGROUND OF THE INVENTION

### 1. Field of the Invention

The invention relates to a povidone-iodine lidocaine gel formulation, and associated methods, allowing for performing a prostate biopsy procedure using the gel formulation.

### 2. Description of the Related Art

Previous studies have strongly supported the use of pre-transrectal ultrasound prostate needle biopsy (TRPNB) bowel prep of povidone-iodine (topical, enema or suppository) to reduce post TRPNB infections.

Prostate cancer is one of the most common malignancies in men. In the United States, 3.1 million cases of prostate cancer were diagnosed between 2003-2017. [Siegel D A, O'Neil M E, Richards T B, et al. Prostate Cancer Incidence and Survival, by Stage and Race/Ethnicity—United States, 2001-2017. MMWR Morb Mortal Wkly Rep 2020; 69:1473-1480]. Prostate biopsy remains the gold standard for diagnosis of prostate cancer, with recent estimates of over 1 million prostate biopsies performed annually in the United States. [Jazayeri S., Kumar J., Nguyen S., et al.] A Systematic Review and Meta-Analysis of Methods Used to Reduce Infectious Complications Following Transrectal Prostate Biopsy, Urology (2020)]. This number will likely increase as the number of patients treated with watchful waiting and active surveillance continues to rise.

While prostate biopsy remains ubiquitous, TRPNB biopsy is not without complications, including dysuria, hematuria, urinary retention, pain/discomfort, and post procedure infection. Post-procedure infectious complications can range from a urinary tract infection to sepsis requiring hospitalization. In addition to adverse patient outcomes, prostate biopsy complications have significant financial costs, with recent estimates totaling $67 million over 2 years only among Medicare patients.

Post-procedural TRPNB infectious complications are likely secondary to seeding of the vascular prostate with bacteria from the rectal vault. While numerous regimens of antibiotic prophylaxis have been described in the literature, the rise of drug resistant organisms, namely fluoroquinolone-resistant E. coli, further complicates peri-procedural infection prevention. Numerous adjustments to peri-procedural and intraprocedural protocols have been described in attempts to mitigate the risk of infectious complications. These countermeasures described in the literature include the novel trans-perineal approach, utilization of pre-procedural rectal swabs to identify patient-specific bacterial resistance patterns, more aggressive and extended antibiotic dosing, utilization of local antibiograms to tailor antibiotic regimens, and intraprocedural needle sterilization.

In addition, multiple studies have detailed utilization of topical antimicrobial preparations, including povidone-iodine solution and suppositories. Compared to other interventions described above, local preparation using povidone-iodine does not incur significant additional costs, nor does it change the standard procedure of the prostate biopsy or pre-biopsy patient preparation in any material way. A seven-year study was performed using only 10% povidone-iodine gel of 787 consecutive transrectal prostate needle biopsy (TRPNB) procedures performed by 2 urologists in a community office and ambulatory surgery center (ASC) setting. For the office procedure, patient's prep included a self-administered saline enema, ciprofloxacin 500 mg p.o. 2 hours before the procedure with additional 500 mg 12 hours and 24 hours following the initial dose. Over the past 2 years, the patients now receive only levofloxacin 750 mg p.o. single dose 2 hours before procedure. All office patients received ultrasound-guided peri-prostatic nerve block in standard fashion.

Patients in the ASC had total intravenous anesthesia with a similar prep and cefazolin 1 gm IV provided concurrently with the procedure and no periprostatic nerve block. They received the same oral antibiotic regimen as described above. Using the povidone-iodine gel only, 1 patient of 787 procedures (0.127%) required hospitalization for treatment of sepsis.

Research has also been conducted regarding the use of lidocaine in TRPNB procedures. For example, see the article “What is the most effective local anesthesia for transrectal ultrasonography-guided biopsy of the prostate? A systematic review and network meta-analysis of 47 randomized clinical trials”, Kim et al. Mar. 20, 2019, Scientific Reports page 1-14.

### SUMMARY OF THE INVENTION

Presented are methods and apparatus for delivering a diagnostic instrument to an organ site within the body of a subject, enabling accurate placement of biopsy needles in areas not directly visible to a surgeon during a procedure. A diagnostic tool is guided to an organ site (using a gel composed of 10% povidone iodine/2% lidocaine) by placing it at a reference site within a body of a subject, the reference site having a known spatial relationship to the organ site, and utilizing a positioning tool to guide a diagnostic tool to a location so positioned with respect to that guiding element that the spatial relationship between that guiding element and that location is substantially similar to the spatial relationship known to exist between the reference site and the organ site, thereby positioning the diagnostic tool substantially at the organ site. Methods and apparatus for imaging at an organ site, and methods and apparatus for performing a systematic or targeted biopsy of an organ are also presented.

It is, therefore, an object of the present invention to provide a combination gel lubricant composition for use in transrectal ultra-sound guided needle biopsy procedures comprising 10% povidone iodine as an antiseptic agent and 2% lidocaine as an anesthetic agent.

It is also an object of the present invention to provide a combination gel lubricant composition including disodium phosphate, glycerin, nonoxynol-10, simethicone, and water.

It is another object of the present invention to provide a method of performing a transrectal ultra-sound guided needle biopsy using the combination gel lubricant composition. The method includes performing a digital rectal exam of a prostate to assess a prostate for abnormalities using the combination gel lubricant composition of 10% povidone iodine and 2% lidocaine, thereby dilating the anus and thereby delivering the combination gel lubricant composition to perianal tissues. The method also includes painting an anterior rectal wall with a fingertip, including a sulci on each lateral prostate border with the combination gel lubricant composition, applying the combination gel lubricant composition to the tip of a transrectal ultrasound transducer probe prior to anal insertion, performing a standard ultra-sound guided bilateral periprostatic nerve block then performing a formal transrectal ultrasound of the prostate to identify hypoechoic areas or irregularities to target as well as calculation of prostate volume. Lastly, ultrasound-guided needle biopsies are obtained in a systematic/targeted fashion using a spring-loaded 18-gauge needle biopsy gun. Approximately 3 to 4 minutes will have elapsed from the initial application of the gel to the rectal tissues and the procurement of the first prostate needle biopsy.

Other objects and advantages of the present invention will become apparent from the following detailed description when viewed in conjunction with the accompanying drawings, which set forth certain embodiments of the invention.

### BRIEF DESCRIPTION OF THE FIGURE

FIG. 1 depicts an ultrasound probe, biopsy gun with needle being inserted into the prostate during a standard biopsy procedure performed at the Mayo Clinic®.

### DETAILED DESCRIPTION OF THE INVENTION

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

A combination gel lubricant composition is used in transrectal ultra-sound guided needle biopsy procedures. As will be disclosed below in greater detail, the combination gel lubricant composition comprises 10% povidone iodine (that is, 10% povidone iodine by weight of the resulting combination gel lubricant composition) as an antiseptic agent and a 2% lidocaine (that is, 2% lidocaine by weight of the resulting combination gel lubricant composition) as an anesthetic agent. The combination gel lubricant composition of 10% povidone-iodine and 2% lidocaine further includes various components known to be used in similar gel compositions, for example, and in addition to the povidone-iodine and lidocaine, the combination gel lubricant composition may include disodium phosphate, glycerin, nonoxynol-10, simethicone, and/or water.

The combination gel lubricant composition of 10% povidone-iodine and 2% lidocaine is used in a method of performing a transrectal ultra-sound guided needle biopsy described below in detail. Briefly, the method includes performing a digital rectal exam of the prostate to assess the prostate for abnormalities using the combination gel lubricant composition of 10% povidone-iodine and 2% lidocaine hydrochloride, thereby dilating the anus and thereby delivering the combination gel lubricant composition of 10% povidone-iodine and 2% lidocaine to perianal tissues. Thereafter, the anterior rectal mucosa, including sulci on each lateral prostate border, is painted with a fingertip using the combination gel lubricant composition of 10% povidone-iodine and 2% lidocaine and the combination gel lubricant composition of 10% povidone-iodine and 2% lidocaine is generously applied to the tip of a transrectal ultrasound probe prior to anal insertion. The medical practitioner then performs a standard ultra-sound guided bilateral periprostatic nerve block and a formal transrectal ultrasound of the prostate to identify hypoechoic areas or irregularities to target as well as calculation of the prostate volume. The ultrasound-guided needle biopsies are then performed sequentially in a systematic and/or targeted fashion securing typically at least 12 cores of tissue

As briefly mentioned above, the combination gel lubricant composition comprises 10% povidone iodine and 2% lidocaine, as one combined product at the time of prostate biopsy, and functions as both an anesthetic and topical antiseptic. In accordance with a preferred embodiment, the remainder of the combination gel lubricant composition is composed of disodium phosphate, glycerin, nonoxynol-10, simethicone, a hydrogel (that is an interlaced network of agar and a copolymer of acrylamide crosslinked with the monomer N, N′-methylene bisacrylamide, and 96 by weight %, bound water), copolymers of 2-hydroxyethyl methacrylate, and/or water in quantities commonly used in other topical gels.

Considering first the povidone iodine, povidone, which is also commonly called polyvinylpyrrolidone, is a synthetic polymer and acts as the antiseptic component. Povidone-iodine (PVP-I) is a complex produced by reacting iodine with the polymer povidone. Aplicare® was a known producer of a Povidone-iodine gel.

Considering now the lidocaine, lidocaine is a synthetic amide, C₁₄H₂₂N₂O, used chiefly in the form of its hydrochloride and acts as the anesthetic. Many known gel materials may be used as the medium for use in conjunction with the lidocaine. For example, it is known to create lidocaine gels by combining lidocaine with a hydrogel that is an interlaced network of agar and a copolymer of acrylamide crosslinked with the monomer N, N′-methylene bisacrylamide, and 96 by weight %, bound water. Other useful examples include gels that are copolymers of 2-hydroxyethyl methacrylate. The water is essential to keep the polymer network expanded, and to disperse the medium. The polymer network serves to hold the liquid in place. The net result is a medium that is transparent, has no fibers to be injected into the tissue, and distributes the medium evenly. Thus, the combined agent will be effective to anesthetize and sterilize the tissue mucosa during the biopsy.

According to another aspect of the present invention, and as briefly discussed above, a method is provided for sampling tissue at an organ site within the body of a subject with the combination gel lubricant composition of 10% povidone-iodine and 2% lidocaine being applied to the tissue and the diagnostic instrument during the procedure.

In particular, the method comprises the following steps that include administering approximately 20 cc of the combination gel lubricant composition of 10% povidone-iodine and 2% lidocaine during the procedure.

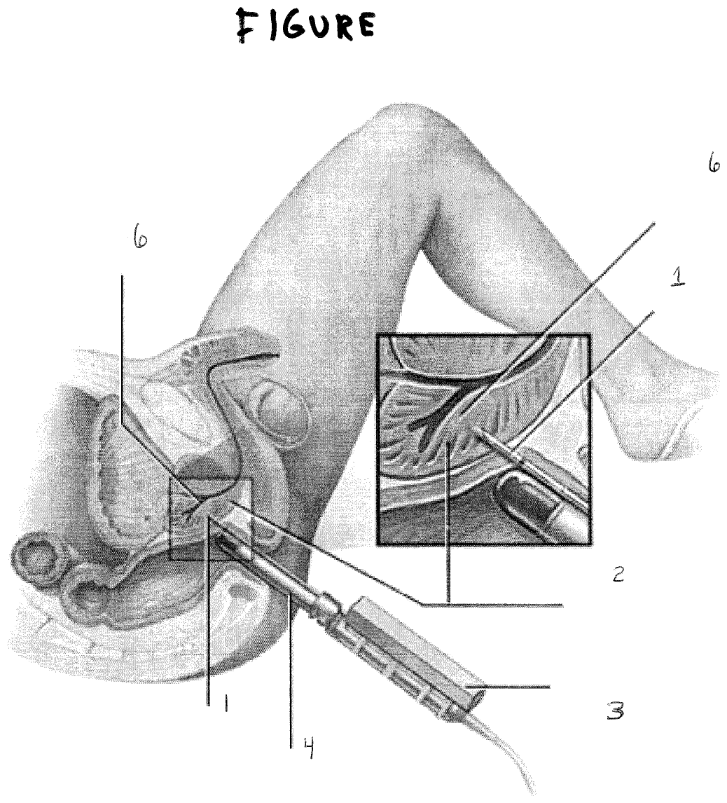

Step One: The patient is placed in a left lateral decubitus position.
Step Two: The combination gel lubricant composition of 10% povidone-iodine and 2% lidocaine is applied to the digit of the medical practitioner and a digital rectal exam prostate is performed to reassess the prostate for abnormalities in such a way that the combination gel lubricant composition of 10% povidone-iodine and 2% lidocaine is spread within the rectum and particularly on the anterior rectal mucosa overlying the prostate.
Step Three: The medical practitioner “paints” with a fingertip the anterior rectal wall, including the sulci on each lateral prostate border, with the combination gel lubricant composition of 10% povidone-iodine and 2% lidocaine.
Step Four: Approximately 15 cc's of the combination gel lubricant composition of 10% povidone-iodine and 2% lidocaine is generously applied to the tip (or head) of a transrectal ultrasound transducer probe prior to anal insertion. A number of assemblies for performing a transrectal ultrasound prostate needle biopsy (TRPNB) are known and FIG. 1 depicts a TRPNB procedure using an assembly including a biopsy gun 3 having a biopsy needle 1 attached thereto, and that apparatus attached to a transrectal ultrasound transducer probe 4.
Step Five: A standard ultra-sound guided bilateral periprostatic nerve block is performed.
Step Six: A formal transrectal ultrasound of the prostate is performed to identify hypoechoic areas or irregularities to target, as well as calculation of the prostate volume.
Step Six: A standard transrectal ultrasound-guided prostate needle biopsy (TRPNB) is then performed in the usual fashion securing 12 biopsy cores or more as appropriate. Briefly, the needle 1 shown in FIG. 1 is inserted into the prostate gland 2 in a template or targeted area 6.

The use of the combination gel lubricant composition of 10% povidone-iodine and 2% lidocaine gel medium at the time of TRPNB provide enhanced anesthetic, antimicrobial and antiseptic benefits during the procedure.

While the invention has been shown and described, it will be understood that there is no intent to limit the invention by such disclosure, but rather, is intended to cover all modifications and alternate constructions falling within the spirit and scope of the invention.

What is claimed is:

1. A combination gel lubricant composition for use in transrectal ultra-sound guided needle biopsy procedures comprising a 10% povidone iodine as an antiseptic agent and 2% lidocaine as an anesthetic agent.

2. The combination gel lubricant composition of claim 1, further comprising disodium phosphate, glycerin, nonoxynol-10, simethicone, and water.

3. The combination gel lubricant composition of claim 2, further comprising sodium carboxymethylcellulose and sodium hydroxide to adjust a pH of the combination gel.

4. A method of performing a transrectal ultra-sound guided needle biopsy using the combination gel lubricant composition as claimed in claim 1, comprising the steps of:
performing a digital rectal exam of a prostate to assess a prostate for abnormalities using the combination gel lubricant composition of 10% povidone iodine and 2% lidocaine, thereby dilating the anus and thereby delivering the combination gel lubricant composition to perianal tissues;
painting an anterior rectal wall, including the sulci on each lateral prostate border with the combination gel lubricant composition;
applying the combination gel lubricant composition to the tip of a transrectal ultrasound transducer probe prior to anal insertion;
performing a standard ultra-sound guided bilateral periprostatic nerve block and performing a formal transrectal ultrasound of the prostate to identify hypoechoic areas or irregularities to target as well as calculation of prostate volume; and
performing the ultrasound-guided needle biopsies of the prostate in systematic and/or targeted fashion.
